(19) United States
(12) Patent Application Publication
(10) Pub. No.: US 2010/0255086 A1
(43) Pub. Date: Oct. 7, 2010

(54) Method for oral mucosal absorption of acetyl salycylic acid

(76) Inventor: Yoel Ovil, Johannesburg (ZA)

Correspondence Address: Dara L. Onofrio, Esq.;c/o ONOFRIO LAW, Suite 225, 15 N. Mill Street, Nyack, NY, 10960, US

(21) Appl. No.: 12/798,469

(22) Filed: Apr. 5, 2010

Related U.S. Application Data
(60) Provisional application No. 61/212,038, filed on Apr. 6, 2009.

(51) Int. Cl. A61K 9/48; A61K 31/616; A61P 9/10; A61P 43/00

(52) U.S. Cl. 424/456; 514/165

(57) ABSTRACT

A method for delivering acetyl salicylic acid to an individual via oral mucosal absorption by providing a melting capsule which includes a therapeutic amount of an acetyl salicylic acid; and administering the capsule sublingually in the patient such that the capsule melts at the individual's body temperature and delivers the acetyl salicylic acid directly into the bloodstream of the individual quickly and without complications; and a melting capsule comprised of a material that has the same melting point as the human body temperature and includes a therapeutic amount of an acetyl salicylic acid liquid or powder having a pH in the range of 2 to 7.

This application claims the benefit of U.S. provisional application No. 61/212,038 filed Apr. 6, 2009, which is incorporated herein in its entirety by reference.

### FIELD OF THE INVENTION

The present invention relates to a method for oral mucosal absorption of acetyl salicylic acid (aspirin). In particular, an aspirin solution is provided in a sublingual melting capsule comprised of a material that has the same melting point as the human body temperature.

### BACKGROUND OF THE INVENTION

Long-term treatment with aspirin can reduce coronary events by 30%-50% due to reduction of platelet aggregation and clot formation. The main draw back of long-term treatment with aspirin is severe gastrointestinal complication (up to 30%) that is associated with high incidence of morbidity and mortality.

Two main mechanisms can cause these serious complications The first, the acid nature of the aspirin further reduce the acidic environment of the stomach; and the second aspirin being an anti-prostaglandin reduces stomach lubrication thus exposing the mucosa to be damaged by the acidic environment.

It is understood that delivering of the aspirin straight from the mouth to the blood bypassing the gastrointestinal system would have major impact on reduction of these life-threatening complications.

The normal pH of the buccal space in humans is approximately 7.3 to 7.4. Buccal absorption of aspirin is only 5%, however we have found that lowering the environmental ph by adding buffer solution had increased notably the absorption rate. At a pH of 4.5 the absorption rate has reached 45%-50% and at pH of 2.5 70% was absorbed,

The present invention provides a method of delivering the aspirin with the appropriate buffer in a form of powder or solution inside a sublingual melting capsule thus reducing and even eliminating the gastrointestinal complications. The buffer component modifies and lowers the pH of acetyl salicylic acid to preferred value of 2.5. This pH change is critical in increasing the absorption rate of the aspirin permitting it to be directly absorbed into the bloodstream when placed in the buccal environment.

In acute myocardial infarction it is essential to reach high levels of aspirin in the blood as soon as possible in order to prevent further damage to the myocardium. Since aspirin does not dissolve in water, it can not be delivered intravenously. Thus, the fastest means to deliver the drug to the blood would be via oral mucosal absorption, preferably sublingually, that is anticipated to be a life saving procedure. The oral mucosal absorption of acetyl salicylic acid not only can reduce notably gastrointestinal complications but also can save life in acute coronary events.

Enterocoated capsules to deliver medications which are absorbed through the gastrointestinal system are an every day routine. However, such conventional delivery is not without its complications and may include gastric bleeding as well as degradation of the medicine by proteolytic enzymes in the gastrointestinal system.

In a simultaneously filed patent application for a medicinal melting capsule, it is described how buccal absorption provides advantages over conventional enterocoated capsules that must be swallowed and pass through the stomach.

The medicinal melting capsule is designed to provide a solution to overcome these problems by melting instantly in the mouth of the individual allowing the inside content to be absorbed directly through the mucosa immediately into the bloodstream without any gastric distress or degradation of the medication.

The prior art has shown various oral and buccal delivery systems for therapeutic agents and active ingredients. See U.S. Pat. Nos. 7,018,653; 6,488,953; 6,183,775; 5,827,525; 5,035,252 and U.S. Patent Publication No. 2008/0317850.

However, none of these prior art systems disclose a melting capsule made of a material that has the same melting point as the human body temperature and includes a therapeutic amount of acetyl salicylic acid which has been manipulated and modified for optimal oral mucosal absorption, preferably sublingual absorption.

The medicinal capsule is designed to overcome the problems of the prior art and melt instantly in the mouth of the individual allowing the inside content to be absorbed directly through the mucosa into the bloodstream.

Advantages of a sublingual delivery over conventional methods include:
1. Faster way to achieve therapeutic blood levels;
2. Bypassing the stomach prevents gastrointestinal complications;
3. Lower medication doses are used to reach the same blood levels delivered using conventional methods;
4. Drug administration via the sublingual method of the invention is possible in unconscious patients without complications;
5. Absorption of the delivered medication is more accurate and predictive and not dependent on stomach content and interference.

Currently, there are very few drugs that are ready as they are, without modification, to be absorbed sublingually. An example of such is nitroglycerine, a tiny molecule that is absorbed instantly.

The medicinal melting capsule allows delivery of medication in optimal form to achieve maximum absorption in the mouth. The use of the medicinal melting capsule is particularly useful in chronic cases where conventional delivery of medications by swallowing would be anticipated to cause stomach irritation bleeding or perforating, i.e. aspirin. The present invention provides an advantage over the known prior art by providing an acetyl salicylic acid solution having a pH between 2-7 contained within a medicinal melting capsule and administered sublingually to a patient to deliver the acetyl salicylic acid directly into the blood stream of the individual quickly and without complications.

### SUMMARY OF THE INVENTION

In the present invention, these purposes, as well as others which will be apparent, are achieved by providing a melting capsule comprised of a material that has the same melting point as the human body temperature and includes a therapeutic amount of an acetyl salicylic acid liquid or powder having a pH between 2-7.

Alternatively, the melting capsule includes a therapeutic amount of an acetyl salicylic acid powder. Preferably the therapeutic amount ranges between 81 to 325 mg per day, but may vary upon desired results.

In preventative applications, the daily dosage is preferably 81 mg. For treatment of acute myocardial infarctions and as a pain killer the recommended daily dosage is preferably 325 mg.

The melting capsule is made of a material that is stable and does not react with the atmosphere or with the acetyl salicylic acid within the capsule wall. The material is selected from the group consisting of chocolate, marshmallow, gelatin, starch, polysaccharide, lipids, proteins and carbohydrates. Preferably the material is chocolate.

Optionally, the melting capsule may contain either an outer layer or inner layer to prevent the capsule from melting before use.

The invention also provides a method for delivering acetyl salicylic acid to an individual via oral mucosal absorption comprising the steps of providing a melting capsule which includes a therapeutic amount of an acetyl salicylic acid; and administering the capsule sublingually in the patient such that the capsule melts at the individual's body temperature and delivers the acetyl acid directly into the bloodstream of the individual quickly and without complications.

The acetyl salicylic acid is in a liquid or powder form. The modified acetyl salicylic acid has a pH in the range of 2 to 7, preferably between 2.5-4.

Other objects, features and advantages of the present invention will be apparent when the detailed description of the preferred embodiments of the invention is considered which should be construed in an illustrative and not limiting sense.

### DETAILED DESCRIPTION OF THE INVENTION

As used herein, the term “mucosal” absorption of a medication and the like is meant to encompass absorption across or through a mucous membrane. In particular, “oral mucosal” absorption of a medication includes delivery across any tissue of the mouth, pharynx, larynx, trachea, particularly including the sublingual, gingival and palatal mucosal tissues.

The term “oral mucosal” absorption as used herein refers to a dosage form wherein medication delivery occurs substantially via the mucosal route and not via swallowing followed by GI absorption. Such dosage forms are designed to provide for a dissolution rate that allows for maximal delivery via the oral mucosa, typically via placement of the dosage form in the sublingual location.

As used herein, “sublingual”, means literally “under the tongue” and refers to a method of administering substances via the mouth in such a way that the substances are rapidly absorbed via the blood vessels under the tongue rather than via the digestive tract. Absorption occurs via highly vascularized buccal mucosa and allows a substance more direct access to the blood circulation, providing for direct systemic administration independent of gastrointestinal influences.

The term aspirin is used interchangeably with its chemical name acetyl salicylic acid. The two terms are meant to mean the same thing.

In accordance with the present invention a melting capsule is provided that contains specially formulated acetyl salicylic acid that is intended to be absorbed directly in the mouth of the individual. The acetyl salicylic acid is modified to have a low pH, preferably around 2.5, and is provided in the capsule in a liquid or powder form which is easily absorbed into the bloodstream.

Different materials are used to manufacture the invention capsules in order to achieve on the one hand maximum protection of the active ingredients and on the other hand to achieve very fast and complete absorption of the medication. In addition the preferred material chosen for such purposes is not harmful and on the contrary it may have benefits and added value.

The invention capsule is manufactured from different melting lipids, carbohydrates and/or proteins including chocolate, marshmallow, gelatin, starch and polysaccharides. These materials have the same melting point as the human body temperature and therefore easily melt when administered orally. In the preferred embodiment the capsule material is chocolate, with or without sugar. In addition, the outer layer of the chocolate capsule can be covered with a polysaccharides layer to protect the chocolate so as not to melt before use, in case the temperature went too high.

Although the capsule can be made of a variety of materials, mentioned above, chocolate is preferred. The capsule when made of a thin layer of chocolate keeps the active material maintained in its optimal condition without interacting with the air light or capsule wall until the moment of usage. The capsule melts instantly when needed and is the optimal way to deliver liquid to the sublingual space. The said liquid is mixed with the chocolate forming an optimal mixture that can adhere to the palate in order to be completely absorbed. Other materials (not chocolate) if mixed with liquid can form inadequate mixture that can lead to catastrophic aspiration especially in an unconscious patient.

In general use of chocolate has many advantages: it is a strong anti oxidant; it reduces LDL and increases HDL; and it is also a strong anti platelet aggregation action and vasodilator. Use of dark, chocolate is most preferred because of its exceptional medicinal qualities and is preferred with diabetic patients.

The use of a chocolate capsule provides two optimal physical properties:

1. The ability to contain liquid as a mini container without interacting or allowing any leakage to occur before clinical usage; and

2. A melting point identical to the body temperature allowing the sublingual melting capsule to melt when it is placed under the tongue.

In addition, the chocolate has a number of biochemical actions that put it in the first line of defense against coronary, cerebro- and peripheral vascular disease. Specifically, chocolate is:

A very potent anti platelet aggregation drug reducing notably the thrombaxane; a strong antioxidant preventing LDL cholesterol to be oxidized; increases levels of HDL (good cholesterol) REF1; and protects endothelial cell function and increases prostacycline secretion.

The concentration of the active ingredient of the acetyl salicylic acid solution is adjusted to be absorbed instantly and completely through the mouth mucosa. Preferably the active ingredient is in the form of a solution, although they can also be in powder form as long as the molecular size is small and/or polar to enhance passive absorption. The active ingredients according to their chemical structure, fat and water solubility, optimal ph and physiological temperature will be dissolved in a solution such that it can be passively absorbed in the mouth to reach therapeutic levels in the blood and if necessary to cross the blood brain barrier.

Acetyl salicylic acid (aspirin) is known to induce gastrointestinal complications. Part of these serious side effects are due to the fact that the aspirin being strong cox inhibitor inhibits prostaglandin formation thus reducing stomach lubrication leading to perforation and bleeding systemic effect), however the aspirin being a strong acid can cause local damage to the gastric mucosa. In an attempt to reduce the said gastric complications aspirin is dissolved and placed into the sublingual melting capsule to be absorbed under the tongue thus achieving complete bypass of the of the gastric mucosa.

In acute myocardial infarction cases when fast action of the aspirin is needed the sublingual melting capsule containing the dissolved aspirin is administrated under the tongue even in unconscious patients to achieve therapeutic blood levels in less than four minutes.

It is obvious that these kinds of patients cannot use the chewable aspirin due to their physical condition. Although use of chewable aspirin can reach optimal blood levels within minutes, such chronic use is also associated with very severe tooth and gum complications that do not occur with the invention mode of administration.

The sublingual melting capsule thus, allows delivery of medication in optimal form to achieve maximum absorption in the mouth, without any adverse effects. The same drug in different solutions, will behave completely differently. The pH and the ratio between unionized to ionized forms (u/I) play a major roll in prediction of membrane permeability.

Henderson-Hasselbalch Equation, which is considered useful for the prediction of membrane permeability of weak acidic and basic drugs and relates the ratio of unionized to ionized forms of drugs to their pka values and pH of the medium could not predict membrane absorption.

In the present invention, a new solution of acetyl salicylic acid, i.e. aspirin, is formed that has a lowered pH between 2 to 4 that leads to swift and effective buccal absorption. The normal pH of the buccal space in humans is approximately 7.3 to 7.4. This solution has been formed with Walpoles acetate buffer (8 ml of acetic acid and 12 ml of sodium acetate solution per 100 ml of water).

The present invention provides a method of delivering the aspirin with the appropriate buffer in a form of powder or solution inside a sublingual melting capsule thus reducing and even eliminating the gastrointestinal complications. The buffer component modifies and lowers the pH of acetyl salicylic acid, which is approximately the same pH as the buccal environment, to preferred value of, 2.5. This pH change is critical in increasing the absorption rate of the aspirin permitting it to be directly absorbed into the bloodstream when placed in the buccal environment.

Testing results have shown that the aspirin solution in a medium of pH 7 had a buccal absorption of 5% whereas the same aspirin solution in a medium of pH 4 had a buccal absorption rate of 45%. Thus, lowering the buffer to pH of 2 (by changing the ratio of acetic acid and sodium acetate) is expected to further increase the absorption rate within the first 4 minutes.

Any other physiological buffer for this purpose can be used in order to achieve optimal acidic environment to enhance the absorption of acetyl salicylic acid solution in the mouth. The consistency of the preferred buffer can also be subjected to changes as needed. In all embodiments, the buffer modifies the acetyl salicylic acid and lowers the pH value of the material below 7 and preferably to around 2.5.

Other non active ingredients can be added as desired, such as binders and flavors.

The invention is designed to treat and prevent a variety of conditions. Preferably the therapeutic amount ranges between 81 to 325 mg per day, but may vary upon desired results. In preventative applications, the daily dosage is preferably 81 mg. For treatment of acute myocardial infarctions and as a pain killer the recommended daily dosage is preferably 325 mg.

Everyday use of aspirin as a prophylactic measure is believed to have reduced coronary events 30% to 50%. Recently it was found not just statistically but also experimentally that the anti-inflammatory action of aspirin plays a major role in prevention of various malignancies.

The main drawback of such long-term prophylaxis treatment with aspirin taken in a conventional manner is the associated gastrointestinal complications. It is understood that two different mechanisms are involved that cause these complications. The first and central mechanism, aspirin being a cox 1 inhibitor decreases production of prostaglandins that in return will decrease gastric lubrication and second and local mechanism, aspirin being an acid itself, reduces the stomach pH further making the stomach environment more acidic and enhancing gastric damage.

Recently the following facts have proven that the local mechanism i.e. the acid nature of the aspirin in touch with the gastric mucosa is the main reason for the gastrointestinal complications. Indomethacin which is also a cox 1 inhibitor (like aspirin) does not cause gastrointestinal complications. Resveratrol, which also inactivates cox 1, is not associated with gastrointestinal complications; and administration of salicylate salts rather than acetyl salicylic acid to the stomach did reduce the gastrointestinal complications by 50%.

It is therefore reasonable to believe that the invention mode of administration of the aspirin sublingually which is absorbed directly in the mouth to the blood stream completely avoiding the gastrointestinal system notably decrease these serious complications, which are the main obstacle in using aspirin as an effective prophylactic tool.

The clinical benefits associated with aspirin treatment in acute myocardial infarction are unprecedented. Within minutes from reaching the blood there is marked reduction of thrombaxane levels leading to immediate inhibition of platelet aggregation preventing further occlusion of the diseased vessel. In addition the aspirin enhances the anaerobic mitochondrial respiration thus improving the energy balance of the involved muscle that its aerobic respiration was interrupted due to lack of oxygen. It is therefore of utmost importance in these conditions to reach high blood levels of acetyl salicylic acid as soon as possible in order to achieve minimal myocardial damage.

Stomach absorption of aspirin tablets is slow due to a variety of factors including:

1. Disintegration of the aspirin tablets (time consuming);

2. Coated aspirin is absorbed in the small intestine alkaline environment which reduces the rate of absorption; and

3. At least thirty two percent of the acetyl salicylic acid reaching the stomach is inactivated there through a deacetylation process;

In an attempt to speed up the absorption rate of aspirin the chewable aspirin was developed. Indeed it is currently the fastest known way today to reach satisfactory therapeutic blood levels of aspirin in acute myocardial infarction, taking approximately seven to ten minutes to enter the blood stream. The main drawback of this system is the absolute need of cooperation of the patient, since the patient must actually chew the tablet. A large number of myocardial infarction patients are unconscious or unable to cooperate which leaves this method of drug delivery to the blood unreliable and unpredicted. In addition, the chewable tablet still passes thru the gastrointestinal system thus causing the complications discussed earlier.

The sublingual melting capsules including the aspirin solution when placed in the individual sublingually will melt even in unconscious patient, thus delivering the aspirin to the blood within three to four minutes, much faster than the chewable aspirin.

Chronic prophylactic use of chewable aspirin was reported to be associated with major teeth and gum problems probably due to the long process of slow release of aspirin in the gum. The instant absorption of the invention aspirin solution will not be associated with these complications.

The foregoing description of various and preferred embodiments of the present invention has been provided for purposes of illustration only, and it is understood that numerous modifications, variations and alterations may be made without departing from the scope and spirit of the invention as set forth in the following claims.

What is claimed is:

1. A melting capsule comprised of a material that has the same melting point as the human body temperature and includes a therapeutic amount of an acetyl salicylic acid liquid or powder having a pH in the range of 2 to 7.

2. The melting capsule of claim 1, wherein said therapeutic amount of said acetyl salicylic acid is in the range of 81 to 325 mg per day.

3. The melting capsule of claim 1, wherein said material is stable and does not react with the atmosphere or with the acetyl salicylic acid within the capsule wall.

4. The melting capsule of claim 1, further containing an outer layer to prevent the capsule from melting before use.

5. The melting capsule of claim 1, further containing an inner layer to prevent the capsule from melting before use.

6. The melting capsule of claim 1, wherein said material is selected from the group consisting of chocolate, marshmallow, gelatin, starch, polysaccharide, lipids, proteins and carbohydrates.

7. The melting capsule of claim 1, wherein said material is chocolate.

8. A melting capsule comprised of a material that has the same melting point as the human body temperature and includes a therapeutic amount of an acetyl salicylic acid powder.

9. A method for delivering acetyl salicylic acid to an individual via oral mucosal absorption comprising the steps of:
providing a melting capsule which includes a therapeutic amount of an acetyl salicylic acid; and
administering the capsule sublingually in the patient such that the capsule melts at the individual's body temperature and delivers the acetyl salicylic acid directly into the bloodstream of the individual quickly and without complications.

10. The method according to claim 8, wherein the acetyl salicylic acid is in a liquid or powder form.

11. The method according to claim 9, wherein the acetyl salicylic acid has a pH in the range of 2 to 7.

12. The method according to claim 9, wherein said therapeutic amount of said acetyl salicylic acid is in the range of 81 to 325 mg per day.
